(19) United States
(12) Patent Application Publication
(10) Pub. No.: US 2008/0095814 A1
(43) Pub. Date: Apr. 24, 2008

(54) Compositions Having a High Antiviral and Antibacterial Efficacy

(75) Inventors: Timothy J. Taylor, Phoenix, AZ (US); Earl P. Seitz Jr., Fountain Hills, AZ (US); Janice L. Fuls, Fountain Hills, AZ (US); George E. Fischler, Phoenix, AZ (US)

Correspondence Address: MARSHALL, GERSTEIN & BORUN LLP (DIAL), 233 S. WACKER DRIVE, 6300 SEARS TOWER, CHICAGO, IL, 60606, US

(73) Assignee: THE DIAL CORPORATION, Scottsdale, AZ (US)

(21) Appl. No.: 11/791,348

(22) PCT Filed: Dec. 5, 2005
(86) PCT No.: PCT/US05/43921

§ 371 Date: Nov. 29, 2007

Related U.S. Application Data
(60) Provisional application No. 60/634,485, filed on Dec. 9, 2004.

(51) Int. Cl. A01N 31/14; A61K 31/075

(52) U.S. Cl. 424/405; 514/721; 514/737

(57) ABSTRACT

Antimicrobial compositions having a rapid antiviral and antibacterial effectiveness are disclosed. The antimicrobial compositions contain a phenolic antimicrobial agent, a surfactant, a hydrotrope, and a disinfecting alcohol, wherein the phenolic antimicrobial agent is present in a continuous aqueous phase in an amount of at least 25% of saturation concentration.

### CROSS REFERENCE TO RELATED APPLICATIONS

This application claims the benefit of U.S. provisional patent application Ser. No. 60/634,485, filed Dec. 9, 2004.

### FIELD OF THE INVENTION

The present invention relates to antimicrobial compositions having a rapid antiviral and antibacterial effectiveness. More particularly, the present invention relates to antimicrobial compositions comprising a phenolic antimicrobial agent, a surfactant, a hydrotrope, and a disinfecting alcohol. The phenolic antimicrobial agent is present in a continuous aqueous phase of the composition in an amount of at least 25% saturation.

### BACKGROUND OF THE INVENTION

Human health is impacted by a variety of microbes encountered on a daily basis. In particular, contact with various microbes in the environment can lead to an illness, possibly severe, in mammals. For example, microbial contamination can lead to a variety of illnesses, including, but not limited to, food poisoning, a streptococcal infection, anthrax (cutaneous), athlete's foot, cold sores, conjunctivitis (“pink eye”), coxsackievirus (hand-foot-mouth disease), croup, diphtheria (cutaneous), ebolic hemorrhagic fever, and impetigo.

It is known that washing body parts (e.g., hand washing) and hard surfaces (e.g., countertops and sinks) can significantly decrease the population of microorganisms, including pathogens. Therefore, cleaning skin and other animate and inanimate surfaces to reduce microbial populations is a first defense in removing such pathogens from these surfaces, and thereby minimizing the risk of infection.

Viruses are one category of pathogens that are of primary concern. Viral infections are among the greatest causes of human morbidity, with an estimated 60% or more of all episodes of human illness in developed countries resulting from a viral infection. In addition, viruses infect virtually every organism in nature, with high virus infection rates occurring among all mammals, including humans, pets, livestock, and zoo specimens.

Viruses exhibit an extensive diversity in structure and lifecycle. A detailed description of virus families, their structures, lifecycles, and modes of viral infection is discussed in Fundamental Virology, 4th Ed., Eds. Knipe & Howley, Lippincott Williams & Wilkins, Philadelphia, Pa., 2001.

Simply stated, virus particles are intrinsic obligate parasites, and have evolved to transfer genetic material between cells and encode sufficient information to ensure their own propagation. In a most basic form, a virus consists of a small segment of nucleic acid encased in a simple protein shell. The broadest distinction between viruses is the enveloped and nonenveloped viruses, i.e., those that do or do not contain, respectively, a lipid-bilayer membrane.

Viruses propagate only within living cells. The principal obstacle encountered by a virus is gaining entry into the cell, which is protected by a cell membrane of thickness comparable to the size of the virus. In order to penetrate a cell, a virus first must become attached to the cell surface. Much of the specificity of a virus for a certain type of cell lies in its ability to attach to the surface of that specific cell. Durable contact is important for the virus to infect the host cell, and the ability of the virus and the cell surface to interact is a property of both the virus and the host cell. The fusion of viral and host-cell membranes allows the intact viral particle, or, in certain cases, only its infectious nucleic acid to enter the cell. Therefore, in order to control a viral infection, it is important to rapidly kill a virus that contacts the skin or a hard surface.

For example, rhinoviruses, influenza viruses, and adenoviruses are known to cause respiratory infections. Rhinoviruses are members of the picornavirus family, which is a family of “naked viruses” that lack an outer envelope. The human rhinoviruses are so termed because of their special adaptation to the nasopharyngeal region, and are the most important etiological agents of the common cold in adults and children. Officially there are 102 rhinovirus serotypes. Most of the picornaviruses isolated from the human respiratory system are acid labile, and this lability has become a defining characteristic of rhinoviruses.

Rhinovirus infections are spread from person to person by direct contact with virus-contaminated respiratory secretions. Typically, this contact is in the form of physical contact with a contaminated surface, rather than via inhalation of airborne viral particles.

Rhinovirus can survive on environmental surfaces for hours after initial contamination, and infection is readily transmitted by finger-to-finger contact, and by contaminated environmental surface-to-finger contact, if the newly contaminated finger then is used to rub an eye or touch the nasal mucosa. Therefore, virus contamination of skin and environmental surfaces should be minimized to reduce the risk of transmitting the infection to the general population.

Several gastrointestinal infections also are caused by viruses. For example, Norwalk virus causes nausea, vomiting (sometimes accompanied by diarrhea), and stomach cramps. This infection typically is spread from person to person by direct contact. Acute hepatitis A viral infection similarly can be spread by direct contact between one infected person and a nonimmune individual by hand-to-hand, hand-to-mouth, or aerosol droplet transfer, or by indirect contact when an uninfected individual comes into contact with a hepatitis A virus-contaminated solid object. Numerous other virus infections are spread similarly. The risk of transmitting such viral infections can be reduced significantly by inactivating or removing viruses from the hands and other environmental surfaces.

Antimicrobial personal care compositions are known in the art. In particular, antibacterial cleansing compositions, which typically are used to cleanse the skin and to destroy bacteria present on the skin, especially the hands, arms, and face of the user, are well-known commercial products.

Antibacterial compositions are used, for example, in the health care industry, food service industry, meat processing industry, and in the private sector by individual consumers. The widespread use of antibacterial compositions indicates the importance consumers place on controlling bacteria populations on skin. The paradigm for antibacterial compositions is to provide a substantial and broad spectrum reduction in bacterial populations quickly and without adverse side effects associated with toxicity and skin irritation. Such antibacterial compositions are disclosed in U.S. Pat. Nos. 6,107,261 and 6,136,771, each incorporated herein by reference.

Virus control poses a more difficult problem, however. By sufficiently reducing bacterial populations, the risk of bacterial infection is reduced to acceptable levels. Therefore, a rapid antibacterial kill is desired. A rapid kill of viruses also is desired. However, in theory, a single virus can cause infection. Therefore, a fast and essentially total antiviral activity is required, or at least desired, for an effective antiviral cleansing composition.

Most commercial antibacterial compositions generally offer a low to moderate antibacterial activity, and no reported antiviral activity. Antibacterial activity is assessed against a broad spectrum of microorganisms, including both Gram positive and Gram negative microorganisms. The log reduction, or alternatively the percent reduction, in bacterial populations provided by the antibacterial composition correlates to antibacterial activity. A 1-3 log reduction is preferred, a log reduction of 3-5 is most preferred, whereas a log reduction of less than 1 is least preferred, for a particular contact time, generally ranging from 15 seconds to 5 minutes. Thus, a highly preferred antibacterial composition exhibits a 3-5 log reduction against a broad spectrum of microorganisms in a short contact time.

It should be noted that high log reductions have been achieved at pH values of 4 and 9, but such log reductions are attributed at least in part to these relatively extreme pH values. Compositions having such extreme pH values can irritate the skin and other surfaces, and, therefore, typically are avoided, especially when composition is not wiped or rinsed from the skin after use. It has been difficult to achieve a high log reduction using an antibacterial composition having a neutral pH of about 5 to about 8.

For example, WO 98/01110 discloses compositions comprising triclosan, surfactants, solvents, chelating agents, thickeners, buffering agents, and water. WO 98/01110 is directed to reducing skin irritation by employing a reduced amount of surfactant.

U.S. Pat. No. 5,635,462 discloses compositions comprising PCMX and selected surfactants. The compositions disclosed therein are devoid of anionic surfactants and nonionic surfactants.

EP 0 505 935 discloses compositions containing PCMX in combination with nonionic and anionic surfactants, particularly nonionic block copolymer surfactants.

WO 95/32705 discloses a mild surfactant combination that can be combined with antibacterial compounds, like triclosan.

WO 95/09605 discloses antibacterial compositions containing anionic surfactants and alkylpolyglycoside surfactants.

WO 98/55096 discloses antimicrobial wipes having a porous sheet impregnated with an antibacterial composition containing an active antimicrobial agent, an anionic surfactant, an acid, and water, wherein the composition has a pH of about 3.0 to about 6.0.

N. A. Allawala et al., J. Amer. Pharm. Assoc.—Sci. Ed., Vol. XLII, no. 5, pp. 267-275 (1953) discusses the antibacterial activity of active antibacterial agents in combination with surfactants.

A. G. Mitchell, J. Pharm. Pharmacol., Vol. 16, pp. 533-537 (1964) discloses compositions containing PCMX and a nonionic surfactant that exhibit antibacterial activity.

U.S. Pat. Nos. 6,107,261 and 6,136,771 disclose highly effective antibacterial compositions. These patents disclose compositions that solve the problem of controlling bacteria on skin and hard surfaces, but are silent with respect to controlling viruses. Applicants are aware of no reference that provides a solution for combating bacteria in a highly effective way, while simultaneously controlling viruses, in the form of a single composition.

Antiviral compositions that inactivate or destroy pathogenic viruses, including rhinovirus, rotavirus, influenza virus, parainfluenza virus, respiratory syncytial virus, and Norwalk virus, also are known. For example, U.S. Pat. No. 4,767,788 discloses the use of glutaric acid to inactivate or destroy viruses, including rhinovirus. U.S. Pat. No. 4,975,217 discloses compositions containing an organic acid and an anionic surfactant, for formulation as a soap or lotion, to control viruses. U.S. Patent Publication 2002/0098159 discloses the use of a proton donating agent and a surfactant, including an antibacterial surfactant, to effect anti-viral and antibacterial properties.

U.S. Pat. No. 6,034,133 discloses a virucidal hand lotion containing malic acid, citric acid, and a C₁₋₆ alcohol. U.S. Pat. No. 6,294,186 discloses combinations of a benzoic acid analog, such as salicyclic acid, and selected metal salts as being effective against viruses, including rhinovirus. U.S. Pat. No. 6,436,885 discloses a combination of known antibacterial agents with 2-pyrrolidone-5-carboxylic acid, at a pH of 2 to 5.5, to provide antibacterial and antiviral properties.

European Patent Application 0 604 848 discloses a gel-type hand disinfectant containing an antimicrobial agent, 40% to 90% by weight of an alcohol, and a polymer and a thickening agent in a combined weight of not more than 3% by weight. The gel is rubbed into the hands and allowed to evaporate to provide disinfected hands. The disclosed compositions often do not provide immediate sanitization and do not provide residual antibacterial efficacy.

Hayden et al., Antimicrobial Agents and Chemotherapy, 26:928-929 (1984), discloses interrupting the hand-to-hand transmission of rhinovirus colds through the use of a hand lotion having a persistent virucidal activity. The hand lotions, containing 2% glutaric acid, were more effective than a placebo in inactivating certain types of rhinovirus. However, the publication discloses that the glutaric acid-containing lotions were not effective against a wide spectrum of rhinovirus serotypes.

A virucidal tissue designed for use by persons infected with the common cold, and including citric acid, malic acid, and sodium lauryl sulfate, is known. Hayden et al., Journal of Infectious Diseases, 152: 493-497 (1985), however, reported that use of paper tissues, either treated with virus-killing substances or untreated, can interrupt the hand-to-hand transmission of viruses. Hence, no distinct advantage in preventing the spread of rhinovirus colds can be attributed to the compositions incorporated into the virucidal tissues.

An efficacious antimicrobial composition effective against both bacteria and viruses has been difficult to achieve because of the fundamental differences between a bacteria and a virus, and because of the properties of the antimicrobial agents and the effects of a surfactant on an antimicrobial agent. For example, several antimicrobial agents, like phenols, have an exceedingly low solubility in water, e.g., triclosan solubility in water is about 5 to 10 ppm (parts per million). The solubility of the antimicrobial agent is increased by adding surfactants to the composition. However, an increase in solubility of the antimicrobial agent, and, in turn, the amount of antimicrobial agent in the composition, does not necessarily lead to an increased efficacy.

Without being bound to any particular theory, it is theorized that the addition of a surfactant increases antimicrobial agent solubility, but also typically reduces the availability of antimicrobial agent because a surfactant in water forms micelles above the critical micelle concentration of the surfactant. The critical micelle concentration varies from surfactant to surfactant. The formation of micelles is important because micelles have a lipophilic region that attracts and solubilizes the antimicrobial agent, which renders the antimicrobial agent unavailable to immediately contact microbes, e.g., bacteria and viruses, and thereby unable to control the microbes in short time period (i.e., one minute or less).

An antimicrobial agent solubilized in the surfactant micelles will control microbes, but in relatively long time frames. The antimicrobial agent, if free in the aqueous solution and not tied up in the surfactant micelle, i.e., is activated, performs its function quickly. If the antimicrobial agent is tied up in the surfactant micelle, i.e., is not activated, the antimicrobial agent is only slowly available and cannot perform its function in a time frame that is practical for cleaning the skin.

In addition, an antimicrobial agent that is solubilized in the micelle is readily washed from the skin during the rinsing process, and is not available to deposit on the skin to provide a persistent antimicrobial benefit. Rather, the antimicrobial agent is washed away and wasted.

Accordingly, a need exists for an antimicrobial composition that is highly efficacious against a broad spectrum of microbes, including viruses and Gram positive and Gram negative bacteria, in a short time period, and is mild to the skin. Cleansing products demonstrating improved mildness and a heightened level of viral and bacterial reduction are provided by the antimicrobial compositions of the present invention.

### SUMMARY OF THE INVENTION

The present invention is directed to antimicrobial compositions that provide a rapid antiviral and antibacterial effectiveness. The compositions provide a substantial viral control and a substantial reduction in Gram positive and Gram negative bacteria in less than about one minute.

More particularly, the present invention relates to antimicrobial compositions containing an active antimicrobial agent, a surfactant, a hydrotrope, a disinfecting alcohol, and water, wherein the antimicrobial agent is present in the continuous aqueous phase (in contrast to being present in micelles) in an amount of at least 25% of saturation, when measured at room temperature.

Accordingly, one aspect of the present invention is to provide an antimicrobial composition that is highly effective at killing a broad spectrum of bacteria, including Gram positive and Gram negative bacteria such as S. aureus, Salmonella choleraesuis, E. coli, and K. pneumoniae, while simultaneously inactivating or destroying viruses harmful to human health, particularly rhinoviruses.

Another aspect of the present invention is to provide a liquid, antimicrobial composition comprising:

(a) about 0.001% to about 5%, by weight, of a phenolic antimicrobial agent;

(b) about 0.1% to 15%, by weight, of a surfactant;

(c) about 2% to about 30%, by weight, of a hydrotrope;

(d) greater than 60% to about 90%, by weight, of a disinfecting alcohol; and

(e) water,

wherein the antimicrobial agent is present in the composition in an amount of at least 25% of saturation concentration, when measured at room temperature.

Another aspect of the present invention is to provide an antimicrobial composition that exhibits a substantial and wide spectrum viral control.

Yet another aspect of the present invention is to provide an antimicrobial composition that exhibits a log reduction against Gram positive bacteria (i.e., S. aureus) of at least 2 after 30 seconds of contact.

Still another aspect of the present invention is to provide an antimicrobial composition that exhibits a log reduction against Gram negative bacteria (i.e., E. coli) of at least 2.5 after 30 seconds of contact.

Another aspect of the present invention is to provide an antimicrobial composition that exhibits a log reduction against viruses, including rhinovirus serotypes, such as Rhinovirus 1a, Rhinovirus 2, Rhinovirus 14, and Rhinovirus 4, of at least 4 after 30 seconds of contact.

Another aspect of the present invention is to provide consumer products based on an antimicrobial composition of the present invention, for example, a skin cleanser, a body splash, a surgical scrub, a wound care agent, a hand sanitizer gel, a disinfectant, a mouth wash, a pet shampoo, a hard surface sanitizer, a lotion, an ointment, a cream, and the like. A composition of the present invention can be a rinse-off product or a leave-on product. Preferably, the composition is allowed to remain on the skin to allow the volatile components of the composition evaporate. The compositions are esthetically pleasing and nonirritating to the skin.

A further aspect of the present invention is to provide a method of quickly controlling a wide spectrum of viruses and Gram positive and/or Gram negative bacteria populations on animal tissue, including human tissue, by contacting the tissue, like the dermis, with a composition of the present invention for a sufficient time, for example, about 15 seconds to 5 minutes or longer, to reduce bacterial and viral population levels to a desired level.

Still another aspect of the present invention is to provide a method treating or preventing virus-mediated diseases and conditions caused by rhinoviruses, adenoviruses, rotaviruses, and similar pathogenic viruses.

Yet another aspect of the present invention is to provide a composition and method of interrupting transmission of a virus from animate and inanimate surfaces to an animate surface, especially human skin.

Especially provided is a method and composition for controlling the transmission of rhinovirus by effectively controlling rhinoviruses present on human skin.

These and other novel aspects and advantages of the present invention are set forth in the following, nonlimiting detailed description of the preferred embodiments.

### DETAILED DESCRIPTION OF THE PREFERRED EMBODIMENTS

Personal care products incorporating an active antimicrobial agent have been known for many years. Since the introduction of antimicrobial personal care products, many claims have been made that such products provide antimicrobial properties. To be most effective, an antimicrobial composition should provide a high log reduction against a broad spectrum of organisms in as short a contact time as possible. Ideally, the composition also should inactivate viruses.

As presently formulated, most commercial liquid antibacterial soap compositions provide a poor to marginal time kill efficacy, i.e., rate of killing bacteria. These compositions do not effectively control viruses.

Most current products especially lack efficacy against Gram negative bacteria, such as E. coli, which are of particular concern to human health. Compositions do exist, however, that have an exceptionally high broad spectrum antibacterial efficacy, as measured by a rapid kill of bacteria (i.e., time kill), which is to be distinguished from a persistent kill. These products also lack a sufficient antiviral activity.

The present antimicrobial compositions provide excellent broad spectrum antibacterial efficacy and significantly improve antiviral efficacy compared to prior compositions. The basis of this improved efficacy is the discovery that the antimicrobial efficacy of an active agent can be correlated to the rate at which the agent has access to an active site on the microbe.

The driving force that determines the rate of antimicrobial agent transport to the site of action is the difference in chemical potential between the site at which the agent acts and the external aqueous phase. Alternatively stated, the microbicidal activity of an active agent is proportional to its thermodynamic activity in the external phase. Accordingly, thermodynamic activity, as opposed to concentration, is the more important variable with respect to antimicrobial efficacy. As discussed more fully hereafter, thermodynamic activity is conveniently correlated to the percent saturation of the active antibacterial agent in the continuous aqueous phase of the composition.

Many compounds have a solubility limit in aqueous solutions, termed the “saturation concentration,” which varies with temperature. Above the saturation concentration, the compound precipitates from solution. Percent saturation is the measured concentration in solution divided by the saturation concentration. The concentration of a compound in aqueous solution can be increased over the saturation concentration in water by the addition of compounds like surfactants. Surfactants not only increase the solubility of compounds in the continuous aqueous phase of the composition, but also form micelles, and can solubilize compounds in the micelles.

The % saturation of an active antimicrobial agent in any composition, including a surfactant-containing composition, ideally can be expressed as:
% saturation=[C/Cₛ]×100%
wherein C is the concentration of antimicrobial agent in solution in the composition and Cₛ is the saturation concentration of the antimicrobial agent in the composition at room temperature. While not wishing to be bound by any theory, applicants believe that the continuous aqueous phase of a surfactant-containing composition is in equilibrium with the micellar pseudophase of said composition, and further that any dissolved species, such as an antimicrobial active agent, is distributed between the aqueous continuous phase and the micellar pseudophase according to a partition law. Accordingly, the percent saturation, or alternatively the relative thermodynamic activity or relative chemical potential, of an antimicrobial active agent dissolved in a surfactant-containing composition is the same everywhere within the composition. Thus, the terms percent saturation of the antimicrobial agent “in a composition,” “in the aqueous continuous phase of a composition,” and “in the micellar pseudophase of a composition” are interchangeable, and are used as such throughout this disclosure.

Maximum antimicrobial efficacy is achieved when the difference in thermodynamic activities of the active antimicrobial agent between the composition and the target organism is maximized (i.e., when the composition is more “saturated” with the active ingredient). A second factor affecting antimicrobial activity is the total amount of available antimicrobial agent present in the composition, which can be thought of as the “critical dose.” It has been found that the total amount of active agent in the continuous aqueous phase of a composition greatly influences the time in which a desired level of antimicrobial efficacy is achieved, given equal thermodynamic activities. Thus, the two key factors affecting the antimicrobial efficacy of an active agent in a composition are: (1) its availability, as dictated by its thermodynamic activity, i.e., percent saturation in the continuous aqueous phase of a composition, and (2) the total amount of available active agent in the solution.

An ingredient in antimicrobial cleansing compositions is a surfactant, which acts as a solubilizer, cleanser, and foaming agent. Surfactants affect the percent saturation of an antimicrobial agent in solution, or more importantly, affect the percent saturation of the active agent in the continuous aqueous phase of the composition. This effect can be explained in the case of a sparingly water-soluble antimicrobial agent in an aqueous surfactant solution, where the active agent is distributed between the aqueous (i.e., continuous) phase and the micellar pseudophase. For antimicrobial agents of exceedingly low solubility in water, such as triclosan, the distribution is shifted strongly toward the micelles (i.e., a vast majority of the triclosan molecules are present in surfactant micelles, as opposed to the aqueous phase).

The ratio of surfactant to antimicrobial agent directly determines the amount of active agent present in the surfactant micelles, which in turn affects the percent saturation of the active agent in the continuous aqueous phase. It has been found that as the surfactant: active agent ratio increases, the number of micelles relative to active molecules also increases, with the micelles being proportionately less saturated with active agent as the ratio increases. Because active agent in the continuous phase is in equilibrium with active agent in the micellar pseudophase, as the saturation of anti-bacterial agent in the micellar phase decreases, so does the saturation of the antimicrobial agent in the continuous phase. The converse also is true. Active agent solubilized in the micellar pseudophase is not immediately available to contact the microorganisms, and it is the percent saturation of active agent in the continuous aqueous phase that determines the antimicrobial activity of the composition. The active agent present in the surfactant micelles, however, can serve as a reservoir of active agent to replenish the continuous aqueous phase as the active agent is depleted.

To summarize, the thermodynamic activity, or percent saturation, of an antimicrobial agent in the continuous aqueous phase of a composition drives antimicrobial activity. Further, the total amount of available active agent determines the ultimate extent of efficacy. In compositions wherein the active agent is solubilized by a surfactant, the active agent present in surfactant micelles is not directly available for antimicrobial activity. For such compositions, the percent saturation of the active agent in the composition, or alternatively the percent saturation of the active agent in the continuous aqueous phase of the composition, determines anti-microbial efficacy.

Although compositions having a high percent saturation of an antimicrobial agent have demonstrated a rapid and effective antibacterial activity against Gram positive and Gram negative bacteria, control of viruses has been inadequate. Virus control on skin and inanimate surfaces is very important in controlling the transmission of numerous diseases.

For example, rhinoviruses are the most significant microorganisms associated with the acute respiratory illness referred to as the “common cold.” Other viruses, such as parainfluenza viruses, respiratory syncytial viruses (RSV), enteroviruses, and coronaviruses, also are known to cause symptoms of the “common cold,” but rhinoviruses are theorized to cause the greatest number of common colds. Rhinoviruses also are among the most difficult of the cold-causing viruses to control, and have an ability to survive on a hard, dry surface for more than four days. In addition, most viruses are inactivated upon exposure to a 70% ethanol solution.

However, rhinoviruses remain viable upon exposure to ethanol.

Because rhinoviruses are the major known cause of the common cold, it is important that a composition having antiviral activity is active against the rhinovirus. Although the molecular biology of rhinoviruses is now understood, finding effective methods of controlling rhinovirus and preventing colds caused by rhinoviruses, and of preventing the spread of the virus to noninfected subjects, has been fruitless.

It is known that iodine is an effective anti-viral agent, and provides a persistent antirhinoviral activity on skin. In experimentally induced and natural cold transmission studies, subjects who used iodine products had significantly fewer colds than placebo users.

This indicates that iodine is effective for prolonged periods at blocking the transmission of rhinoviral infections. Thus, the development of products that provide antiviral activity would be effective in reducing the incidents of colds. Likewise, a topically applied composition that exhibits antiviral activity would be effective in preventing and/or treating diseases caused by other pathogenic viruses.

The antimicrobial compositions of the present invention are highly effective in providing a rapid and broad spectrum control of bacteria, and a rapid and broad spectrum control of viruses, i.e., are virucidal. Virucidal means capable of inactivating or destroying a virus. The present compositions are highly effective and comprise a high percent saturation concentration of a phenolic antimicrobial agent in a phase stable formulation. The compositions are surprisingly mild to the skin, and noncorrosive to inanimate surfaces. Thus, mild and effective compositions that solve the problem of bacterial and viral control are provided to consumers.

The antimicrobial compositions of the present invention are highly efficacious in household cleaning applications (e.g., hard surfaces, like floors, countertops, tubs, dishes, and softer cloth materials, like clothing), personal care applications (e.g., lotions, shower gels, soaps, shampoos, and wipes), and industrial and hospital applications (e.g., sterilization of instruments, medical devices, and gloves). The present compositions efficaciously and rapidly clean and disinfect surfaces that are infected or contaminated with Gram negative bacteria, Gram positive bacteria, and viruses (e.g., rhinoviruses).

The present compositions can be used in vitro and in vivo. In vitro means in or on nonliving things, especially on inanimate objects having hard or soft surfaces located or used where preventing viral transmission is desired, most especially on objects that are touched by human hands. In vivo means in or on animate objects, especially on mammal skin, and particularly on hands.

As illustrated in the following nonlimiting embodiments, an antimicrobial composition of the present invention comprises: (a) about 0.001% to about 5%, by weight, of a phenolic antimicrobial agent; (b) about 0.1% to about 15%, by weight, of a surfactant; (c) about 2% to about 30%, by weight, of a hydrotrope; (d) greater than 60% to about 90%, by weight, of a disinfecting alcohol; and (e) water. The compositions have a percent saturation of antimicrobial agent in the continuous aqueous phase of at least about 25%, when measured at room temperature.

The compositions exhibit a log reduction against Gram positive bacteria of about 2 after 30 seconds contact. The compositions also exhibit a log reduction against Gram negative bacteria of about 2.5 after 30 seconds contact. The compositions further exhibit a log reduction against viruses, including rhinovirus serotypes, of about 4 after 30 seconds contact.

In accordance with the invention, a present antimicrobial composition can further comprise additional optional ingredients disclosed hereafter, like polyhydric solvents, pH adjusters, dyes, skin conditioners, and perfumes.

The following ingredients are present in an antimicrobial composition of the present invention.

A. Antimicrobial Agent

An antimicrobial agent is present in a composition of the present invention in an amount of about 0.001% to about 5%, and preferably about 0.01% to about 2%, by weight of the composition. To achieve the full advantage of the present invention, the antimicrobial agent is present in an amount of about 0.05% to about 1%, by weight of the composition.

The antimicrobial compositions can be ready to use compositions, which typically contain 0.001% to about 2%, preferably 0.01% to about 1.5%, and most preferably about 0.05% to about 1%, of an antimicrobial agent, by weight of the composition. The antimicrobial compositions also can be formulated as concentrates that are diluted before use with one to about 50 parts water to provide an end use composition. The concentrated compositions typically contain greater than about 0.05% and up to about 5%, by weight, of the antimicrobial agent. Applications also are envisioned wherein the end use composition contains greater than 2%, by weight, of the antimicrobial agent.

As discussed above, the absolute amount of antimicrobial agent present in the composition is not as important as the amount of available antimicrobial agent in the composition. The amount of available antimicrobial agent in the composition is related to the identity of the surfactant in the composition, the amount of surfactant in the composition, and the presence of optional ingredients in the composition.

To achieve the desired bacteria kill in a short contact time, like 15 to 60 seconds, the continuous aqueous phase of the composition contains an amount of antimicrobial agent that is at least about 25%, preferably at least about 50%, and more preferably at least about 75%, of the saturation concentration of the anti-microbial agent in water, when measured at room temperature. To achieve the full advantage of the present invention, the continuous aqueous phase is about 95% to 100% saturated with the antimicrobial agent. The amount of antibacterial agent present in the continuous aqueous phase can be defined as the total amount of antimicrobial agent in the composition, less any antimicrobial agent present in surfactant micelles. The method of determining percent saturation of antibacterial agent in the composition is disclosed hereafter.

The antimicrobial agents useful in the present invention are phenolic compounds exemplified by the following classes of compounds:

(a) 2-Hydroxydiphenyl Compounds

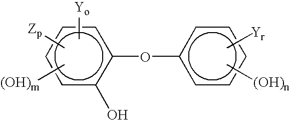

wherein Y is chlorine or bromine, Z is SO₃H, NO₂, or C₁-C₄ alkyl, r is 0 to 3, o is 0 to 3, p is 0 or 1, m is 0 or 1, and n is 0 or 1.

In preferred embodiments, Y is chlorine or bromine, m is 0, n is 0 or 1, o is 1 or 2, r is 1 or 2, and p is 0.

In especially preferred embodiments, Y is chlorine, m is 0, n is 0, o is 1, r is 2, and p is 0.

A particularly useful 2-hydroxydiphenyl compound has a structure: 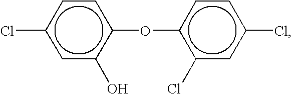
having the adopted name, triclosan, and available commercially under the tradename IRGASAN IP300, from Ciba Specialty Chemicals Corp., Greensboro, N.C. Another useful 2-hydroxydiphenyl compound is 2,2′-dihydroxy-5,5′-dibromo-diphenyl ether.

(b) Phenol Derivatives

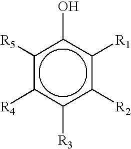

wherein R₁ is hydro, hydroxy, C₁-C₄ alkyl, chloro, nitro, phenyl, or benzyl; R₂ is hydro, hydroxy, C₁-C₆ alkyl, or halo; R₃ is hydro, C₁-C₆ alkyl, hydroxy, chloro, nitro, or a sulfur in the form of an alkali metal salt or ammonium salt; R₄ is hydro or methyl; and R₅ is hydro or nitro. Halo is bromo or, preferably, chloro.

Specific examples of phenol derivatives include, but are not limited to, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexylresorcinol, pyrogallol, phloroglucin, carvacrol, thymol, p-chlorothymol, o-phenylphenol, o-benzylphenol, p-chloro-o-benzylphenol, phenol, 4-ethylphenol, and 4-phenolsulfonic acid. Other phenol derivatives are listed in U.S. Pat. No. 6,436,885, incorporated herein by reference.

(c) Diphenyl Compounds

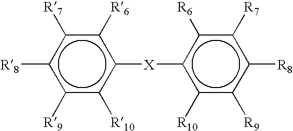

wherein X is sulfur or a methylene group, R₆ and R′₆ are hydroxy, and R₇, R′₇, R₈, R′₈, R₉, R′₉, R₁₀, and R′₁₀, independent of one another, are hydro or halo. Specific, nonlimiting examples of diphenyl compounds are hexachlorophene, tetrachlorophene, dichlorophene, 2,3-dihydroxy-5,5′-dichlorodiphenyl sulfide, 2,2′-dihydroxy-3,3′,5,5′-tetrachlorodiphenyl sulfide, 2,2′-dihydroxy-3,5′,5,5′,6,6′-hexachlorodiphenyl sulfide, and 3,3′-dibromo-5,5′-dichloro-2,2′-dihydroxydiphenylamine. Other diphenyl compounds are listed in U.S. Pat. No. 6,436,885, incorporated herein by reference.

B. Surfactant

In addition to the antimicrobial agent, a present antimicrobial composition also contains a surfactant. The surfactant is present in an amount of about 0.1% to about 15%, and preferably about 0.3% to about 10%, by weight of the composition. To achieve the full advantage of the present invention, the antimicrobial composition contains about 0.5% to about 7%, by weight, of the surfactant.

Ready-to-use compositions typically contain about 0.1% to about 10% of a surfactant, preferably about 0.3% to about 5%, and most preferably, 0.5% to about 3%, by weight of the composition. Concentrated compositions suitable for dilution typically contain greater than about 5%, by weight, of a surfactant.

The amount of surfactant present in the composition is related to the amount and identity of the anti-microbial agent in the composition and to the identity of the surfactant. The amount of surfactant is determined such that the percent saturation of the antimicrobial agent in the continuous aqueous phase of the composition is at least about 25%, preferably at least about 50%, more preferably at least about 75%, and most preferably at least about 95%.

The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition. Anionic surfactants are preferred.

The antimicrobial compositions, therefore, can contain an anionic surfactant having a hydrophobic moiety, such as a carbon chain including about 8 to about 30 carbon atoms, and particularly about 12 to about 20 carbon atoms, and further has a hydrophilic moiety, such as sulfate, sulfonate, carbonate, phosphate, or carboxylate. Often, the hydrophobic carbon chain is etherified, such as with ethylene oxide or propylene oxide, to impart a particular physical property, such as increased water solubility or reduced surface tension to the anionic surfactant.

Suitable anionic surfactants include, but are not limited to, compounds in the classes known as alkyl sulfates, alkyl ether sulfates, alkyl ether sulfonates, sulfate esters of an alkylphenoxy polyoxyethylene ethanol, alpha-olefin sulfonates, beta-alkoxy alkane sulfonates, alkylaryl sulfonates, alkyl monoglyceride sulfates, alkyl monoglyceride sulfonates, alkyl carbonates, alkyl ether carboxylates, fatty acids, sulfosuccinates, sarcosinates, octoxynol or nonoxynol phosphates, taurates, fatty taurides, fatty acid amide polyoxyethylene sulfates, isethionates, acyl glutamates, alkyl sulfoacetates, acylated peptides, acyl lactylates, anionic fluoro surfactants, and mixtures thereof. Additional anionic surfactants are listed in McCutcheon's Emulsifiers and Detergents, 1993 Annuals, (hereafter McCutcheon's), McCutcheon Division, MC Publishing Co., Glen Rock, N.J., pp. 263-266, incorporated herein by reference. Numerous other anionic surfactants, and classes of anionic surfactants, are disclosed in U.S. Pat. No. 3,929,678 and U.S. Patent Publication No. 2002/0098159, each incorporated herein by reference.

Specific, nonlimiting classes of anionic surfactants useful in the present invention include, but are not limited to, a C₈-C₁₈ alkyl sulfonate, a C₈-C₁₈ alkyl sulfate, a C₈-C₁₈ fatty acid salt, a C₈-C₁₈ alkyl ether sulfate having one or two moles of ethoxylation, a C₈-C₁₈ alkamine oxide, a C₈-C₁₈ alkoyl sarcosinate, a C₈-C₁₈ sulfoacetate, a C₈-C₁₈ sulfosuccinate, a C₈-C₁₈ alkyl diphenyl oxide disulfonate, a C₈-C₁₈ alkyl carbonate, a C₈-C₁₈ alpha-olefin sulfonate, a methyl ester sulfonate, and mixtures thereof. The C₈-C₁₈ alkyl group contains eight to eighteen carbon atoms, and can be straight chain (e.g., lauryl) or branched (e.g., 2-ethylhexyl). The cation of the anionic surfactant can be an alkali metal (preferably sodium or potassium), ammonium, C₁-C₄ alkylammonium (mono-, di-, tri-), or C₁-C₃ alkanolammonium (mono-, di-, tri-). Lithium and alkaline earth cations (e.g., magnesium) can be used, but are not preferred.

Specific surfactants include, but are not limited to, lauryl sulfates, octyl sulfates, 2-ethylhexyl sulfates, decyl sulfates, tridecyl sulfates, cocoates, lauroyl sarcosinates, lauryl sulfosuccinates, linear C₁₀ diphenyl oxide disulfonates, lauryl sulfosuccinates, lauryl ether sulfates (1 and 2 moles ethylene oxide), myristyl sulfates, oleates, stearates, tallates, ricinoleates, cetyl sulfates, and similar surfactants. Additional examples of surfactants can be found in “CTFA Cosmetic Ingredient Handbook,” J. M. Nikitakis, ed., The Cosmetic, Toiletry and Fragrance Association, Inc., Washington, D.C. (1988) (hereafter CTFA Handbook), pages 10-13, 42-46, and 87-94, incorporated herein by reference.

The antimicrobial compositions also can contain nonionic surfactants. Typically, a nonionic surfactant has a hydrophobic base, such as a long chain alkyl group or an alkylated aryl group, and a hydrophilic chain comprising a sufficient number (i.e., 1 to about 30) of ethoxy and/or propoxy moieties. Examples of classes of nonionic surfactants include ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, and mixtures thereof.

Exemplary nonionic surfactants include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG 600 dioleate, PEG 400 dioleate, and mixtures thereof.

Numerous other nonionic surfactants are disclosed in McCutcheon's at pages 1-246 and 266-272; in the CTFA International Cosmetic Ingredient Dictionary, Fourth Ed., Cosmetic, Toiletry and Fragrance Association, Washington, D.C. (1991) (hereinafter the CTFA Dictionary) at pages 1-651; and in the CTFA Handbook, at pages 86-94, each incorporated herein by reference.

In addition to anionic and nonionic surfactants, cationic, ampholytic, and amphoteric surfactants can be used in the present antimicrobial compositions. Useful cationic surfactants include those having a structural formula

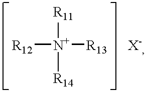

wherein R₁₁ is an alkyl group having about 12 to about 30 carbon atoms, or an aromatic, aryl, or alkaryl group having about 12 to about 30 carbon atoms; R₁₂, R₁₃, and R₁₄, independently, are selected from the group consisting of hydrogen, an alkyl group having 1 to about 22 carbon atoms, or aromatic, aryl, or alkaryl groups having from about 12 to about 22 carbon atoms; and X is a compatible anion, preferably selected from the group consisting of chloride, bromide, iodide, acetate, phosphate, nitrate, sulfate, methyl sulfate, ethyl sulfate, tosylate, lactate, citrate, glycolate, and mixtures thereof. Additionally, the alkyl groups of R₁₁, R₁₂, R₁₃, and R₁₄ also can contain ester and/or ether linkages, or hydroxy or amino group substituents (e.g., the alkyl groups can contain polyethylene glycol and polypropylene glycol moieties).

Preferably, R₁₁ is an alkyl group having about 12 to about 22 carbon atoms; R₁₂ is H or an alkyl group having 1 to about 22 carbon atoms; and R₁₃ and R₁₄, independently are H or an alkyl group having 1 to about 3 carbon atoms. More preferably, R₁₁ is an alkyl group having about 12 to about 22 carbon atoms, and R₁₂, R₁₃, and R₁₄ are H or an alkyl group having 1 to about 3 carbon atoms.

Other useful cationic surfactants include amino-amides, wherein in the above structure R₁₁ alternatively is R₁₅CONH—(CH₂)ₙ, wherein R₅ is an alkyl group having about 12 to about 22 carbon atoms, and n is an integer of 2 to 6, more preferably 2 to 4, and most preferably 2 to 3. Nonlimiting examples of these cationic surfactants include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Nonlimiting examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof.

Additional quaternary ammonium salts include those wherein the C₁₂-C₃₀ alkyl carbon chain is derived from a tallow fatty acid or from a coconut fatty acid. The term “tallow” refers to an alkyl group derived from tallow fatty acids (usually hydrogenated tallow fatty acids), which generally has mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, and mixtures thereof. An example of a quaternary ammonium compound having an alkyl group with an ester linkage is ditallowyl oxyethyl dimethyl ammonium chloride.

Ampholytic surfactants, i.e., amphoteric and zwitterionic surfactants, can be broadly described as derivatives of secondary and tertiary amines having straight chain or branched aliphatic radicals, and wherein one of the aliphatic substituents contains from about 8 to about 18 carbon atoms and at least one of the aliphatic substituents contains an anionic water-solubilizing group, e.g., carboxy, sulfonate, or sulfate.

More particularly, one class of ampholytic surfactants include sarcosinates and taurates having the general structural formula 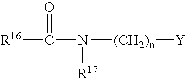
wherein R¹⁶ is C₁₁ through C₂₁ alkyl, R¹⁷ is hydrogen or C₁-C₂ alkyl, Y is CO₂M or SO₃M, M is an alkali metal, and n is a number 1 through 3.

Another class of ampholytic surfactants is the amide sulfosuccinates having the structural formula

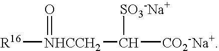

The following classes of ampholytic surfactants also can be used: 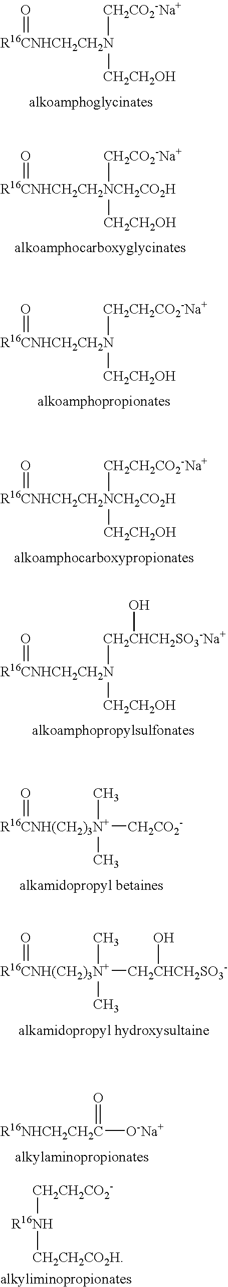
Additional classes of ampholytic surfactants include the phosphobetaines and the phosphitaines.

Specific, nonlimiting examples of ampholytic surfactants useful in the present invention are sodium coconut N-methyl taurate, sodium oleyl N-methyl taurate, sodium tall oil acid N-methyl taurate, sodium palmitoyl N-methyl taurate, cocodimethylcarboxymethylbetaine, lauryldimethylcarboxymethylbetaine, lauryldimethylcarboxyethylbetaine, cetyldimethylcarboxymethylbetaine, lauryl-bis-(2-hydroxyethyl carboxymethylbetaine, oleyldimethylgammacarboxypropylbetaine, lauryl-bis-(2-hydroxypropyl)-carboxyethylbetaine, cocoamidodimethylpropylsultaine, stearylamidodimethylpropylsultaine, laurylamidobis-(2-hydroxyethyl)propylsultaine, disodium oleamide PEG-2 sulfosuccinate, TEA oleamido PEG-2 sulfosuccinate, disodium oleamide MEA sulfosuccinate, disodium oleamide MIPA sulfosuccinate, disodium ricinoleamide MEA sulfosuccinate, disodium undecylenamide MEA sulfosuccinate, disodium wheat germamido MEA sulfosuccinate, disodium wheat germamido PEG-2 sulfosuccinate, disodium isostearamideo MEA sulfosuccinate, cocoamphoglycinate, cocoamphocarboxyglycinate, lauroamphoglycinate, lauroamphocarboxyglycinate, capryloamphocarboxyglycinate, cocoamphopropionate, cocoamphocarboxypropionate, lauroamphocarboxypropionate, capryloamphocarboxypropionate, dihydroxyethyl tallow glycinate, cocamido disodium 3-hydroxypropyl phosphobetaine, lauric myristic amido disodium 3-hydroxypropyl phosphobetaine, lauric myristic amido glyceryl phosphobetaine, lauric myristic amido carboxy disodium 3-hydroxypropyl phosphobetaine, cocoamido propyl monosodium phosphitaine, lauric myristic amido propyl monosodium phosphitaine, and mixtures thereof.

Useful amphoteric surfactants also include the amine oxides. Amine oxides have a general structural formula wherein the hydrophilic portion contains a nitrogen atom that is bound to an oxygen atom with a semipolar bond. 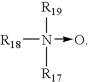
R₁₇, R₁₈, and R₁₉ can be a saturated or unsaturated, branched, or unbranched alkyl or alkenyl group having 1 to about 24 carbon atoms. Preferred amine oxides contain at least one R group that is an alkyl chain of 8 to 22 carbon atoms. Nonlimiting examples of amine oxides include alkyl dimethyl amine oxides, such as decylamine oxide, cocamine oxide, myristamine oxide, and palmitamine oxide. Also useful are the alkylaminopropylamineoxides, for example, coamidopropylamine oxide and stearamidopropylamine oxide.

Nonlimiting examples of preferred surfactants utilized in a present antimicrobial composition include those selected from the group consisting of alkyl sulfates; alkyl ether sulfates; alkyl benzene sulfonates; alpha olefin sulfonates; primary or secondary alkyl sulfonates; alkyl phosphates; acyl taurates; alkyl sulfosuccinates; alkyl sulfoacetates; sulfonated fatty acids; alkyl trimethyl ammonium chlorides and bromides; dialkyl dimethyl ammonium chlorides and bromides; alkyl dimethyl amine oxides; alkylamidopropyl amine oxides; alkyl betaines; alkyl amidopropyl betaines; and mixtures thereof. More preferred surfactants include those selected from the group consisting of alkyl sulfates; alkyl ether sulfates; alkyl benzene sulfonates; alpha olefin sulfonates; primary or secondary alkyl sulfonates; alkyl dimethyl amine oxides; alkyl betaines; and mixtures thereof.

C. Hydrotrope

In addition to the antimicrobial agent and surfactant, a present antimicrobial composition contains a hydrotrope. A hydrotrope is present in an amount of about 2% to about 30%, and preferably about 5% to about 20%, by weight of the composition. To achieve the full advantage of the present invention, a composition contains about 7% to about 15%, by weight, of a hydrotrope.

A hydrotrope is a compound that has an ability to enhance the water solubility of other compounds. A hydrotrope utilized in the present invention lacks surfactant properties, and typically is a short-chain alkyl aryl sulfonate. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

D. Disinfecting Alcohol

The antimicrobial compositions of the present invention contain greater than 60% to about 90%, by weight, of a disinfecting alcohol. Preferred embodiments of the present invention contain about 62% to about 85%, by weight, of a disinfecting alcohol. Most preferred embodiments contain about 65% to about 80%, by weight, of a disinfecting alcohol.

As used herein, the term “disinfecting alcohol” is a water-soluble alcohol containing one to six carbon atoms. Disinfecting alcohols include, but are not limited to, methanol, ethanol, propanol, and isopropyl alcohol.

E. Carrier

The carrier of the present antimicrobial composition comprises water.

F. Optional Ingredients

An antimicrobial composition of the present invention also can contain optional ingredients well known to persons skilled in the art. The optional ingredients are present in a sufficient amount to perform their intended function and not adversely affect the antimicrobial efficacy of the composition. Optional ingredients typically are present, individually, from 0% to about 5%, by weight of the composition, and, collectively, from 0% to about 20%, by weight of the composition.

Classes of optional ingredients include, but are not limited to, polyhydric solvents, dyes, fragrances, pH adjusters, thickeners, viscosity modifiers, chelating agents, skin conditioners, emollients, preservatives, buffering agents, foam stabilizers, antioxidants, foam enhancers, chelating agents, opacifiers, and similar classes of optional ingredients known to persons skilled in the art.

Specific classes of optional ingredients include alkanolamides as foam boosters and stabilizers; gums and polymers as thickening agents; inorganic phosphates, sulfates, and carbonates as buffering agents; EDTA and phosphates as chelating agents; and acids and bases as pH adjusters.

A polyhydric solvent, if present at all, is present in an amount of about 0.1% to about 20%, and preferably about 5% to about 20%, by weight of the composition. To achieve the full advantage of the present invention, the polyhydric solvent is present in an amount of about 10% to about 20% by weight of the composition. In contrast to a disinfecting alcohol, a polyhydric solvent contributes minimally, if at all, to the antimicrobial efficacy of a present composition.

A polyhydric solvent is a water-soluble organic compound containing two to six, and typically two or three, hydroxyl groups. The term “water-soluble” means that the polyhydric solvent has a water solubility of at least 0.1 g of polyhydric solvent per 100 g of water at 25° C. There is no upper limit to the water solubility of the polyhydric solvent, e.g., the polyhydric solvent and water can be soluble in all proportions.

The term polyhydric solvent, therefore, encompasses water-soluble diols, triols, and polyols. Specific examples of polyhydric solvents include, but are not limited to, ethylene glycol, propylene glycol, glycerol, diethylene glycol, dipropylene glycol, tripropylene glycol, hexylene glycol, butylene glycol, 1,2,6-hexanetriol, sorbitol, PEG-4, and similar polyhydroxy compounds.

Examples of preferred classes of optional basic pH adjusters are ammonia; mono-, di-, and tri-alkyl amines; mono-, di-, and tri-alkanolamines; alkali metal and alkaline earth metal hydroxides; and mixtures thereof. However, the identity of the basic pH adjuster is not limited, and any basic pH adjuster known in the art can be used. Specific, nonlimiting examples of basic pH adjusters are ammonia; sodium, potassium, and lithium hydroxide; monoethanolamine; triethylamine; isopropanolamine; diethanolamine; and triethanolamine.

Examples of preferred classes of optional acidic pH adjusters are the mineral acids. Nonlimiting examples of mineral acids are hydrochloric acid, nitric acid, phosphoric acid, and sulfuric acid. The identity of the acidic pH adjuster is not limited and any acidic pH adjuster known in the art, alone or in combination, can be used.

An optional alkanolamide to provide composition thickening, foam enhancement, and foam stability can be, but is not limited to, cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallow-amide MEA, isostearamide DEA, isostearamide MEA, and mixtures thereof.

G. pH

The pH of a present antimicrobial composition is about 4 to about 9 at 25° C., but at the two extremes of this pH range, the compositions may be irritating to the skin or damaging to other surfaces contacted by the composition. Accordingly, antimicrobial compositions of the present invention preferably have a pH of about 5 to about 8, and more preferably about 6 to about 5 to about 8, and more preferably about 6 to about 8. To achieve the full advantage of the present invention, the antimicrobial compositions have a pH of about 6.5 to about 7.5.

To demonstrate the new and unexpected results provided by the antimicrobial compositions of the present invention, the following example is prepared, and the ability of the composition to control Gram positive and Gram negative bacteria, and to control rhinovirus, is determined. The weight percentage listed in the example represents the actual, or active, weight amount of each ingredient present in the composition. The composition is prepared by blending the ingredients, as understood by those skilled in the art and as described below.

The following methods are used in the preparation and testing of the example:

a) Determination of Rapid Germicidal (Time Kill) Activity of Antibacterial Products. The activity of antibacterial compositions is measured by the time kill method, whereby the survival of challenged organisms exposed to an antibacterial test composition is determined as a function of time. In this test, a diluted aliquot of the composition is brought into contact with a known population of test bacteria for a specified time period at a specified temperature. The test composition is neutralized at the end of the time period, which arrests the antibacterial activity of the composition. The percent or, alternatively, log reduction from the original bacteria population is calculated.

In general, the time kill method is known to those skilled in the art.

The composition can be tested at any concentration up to 100%. The choice of which concentration to use is at the discretion of the investigator, and suitable concentrations are readily determined by those skilled in the art. For example, viscous samples usually are tested at 50% dilution, whereas nonviscous samples are not diluted. The test sample is placed in a sterile 250 ml beaker equipped with a magnetic stirring bar and the sample volume is brought to 100 ml, if needed, with sterile deionized water. All testing is performed in triplicate, the results are combined, and the average log reduction is reported.

The choice of contact time period also is at the discretion of the investigator. Any contact time period can be chosen. Typical contact times range from 15 seconds to 5 minutes, with 30 seconds and 1 minute being typical contact times. The contact temperature also can be any temperature, typically room temperature, or about 25 degrees Celsius.

The bacterial suspension, or test inoculum, is prepared by growing a bacterial culture on any appropriate solid media (e.g., agar). The bacterial population then is washed from the agar with sterile physiological saline and the population of the bacterial suspension is adjusted to about 10⁸ colony forming units per ml (cfu/ml).

The table below lists the test bacterial cultures used in the tests and includes the name of the bacteria, the ATCC (American Type Culture Collection) identification number, and the abbreviation for the name of the organism used hereafter. S. aureus is a Gram positive bacteria, whereas E. coli, K. pneum, and S. choler. are Gram negative bacteria.

| Organism Name | ATCC # | Abbreviation |
|---|---|---|
| Staphylococcus aureus | 6538 | S. aureus |
| Escherichia coli | 11229 | E. coli |
| Klebsiella pneumoniae | 10031 | K. pneum. |
| Salmonella choleraesuis | 10708 | S. choler. |

The beaker containing the test composition is placed in a water bath (if constant temperature is desired), or placed on a magnetic stirrer (if ambient laboratory temperature is desired). The sample then is inoculated with 1.0 ml of the test bacteria suspension.

The inoculum is stirred with the test composition for the predetermined contact time. When the contact time expires, 1.0 ml of the test composition/bacteria mixture is transferred into 9.0 ml of Neutralizer Solution. Decimal dilutions to a countable range then are made. The dilutions can differ for different organisms. Selected dilutions are plated in triplicate on TSA+ plates (TSA+ is Trypticase Soy Agar with Lecithin and Polysorbate 80). The plates then are incubated for 24±2 hours, and the colonies are counted for the number of survivors and the calculation of percent or log reduction. The control count (numbers control) is determined by conducting the procedure as described above with the exception that deionized water is used in place of the test composition. The plate counts are converted to cfu/ml for the numbers control and samples, respectively, by standard microbiological methods.

The log reduction is calculated using the formula
Log reduction=log₁₀ (numbers controlled)−log₁₀ (test sample survivors).

The following table correlates percent reduction in bacteria population to log reduction:

| % Reduction | Log Reduction |
|---|---|
| 90 | 1 |
| 99 | 2 |
| 99.9 | 3 |
| 99.99 | 4 |
| 99.999 | 5 |

b) Antiviral Efficacy Test

References: S. A. Sattar, Standard Test Method for Determining the Virus-Eliminating Effectiveness of Liquid Hygenic Handwash Agents Using the Fingerpads of Adult Volunteers, Annual Book of ASTM Standards. Designation E1838-96, incorporated herein by reference in its entirety, and referred to as “Sattar I”; and S. A. Sattar et al., Chemical Disinfection to Interrupt Transfer of Rhinovirus Type 14 from Environmental Surfaces to Hands, Applied and Environmental Microbiology, Vol. 59, No. 5, May, 1993, pp. 1579-1585, incorporated herein by reference in its entirety, and referred to as “Sattar II.”

The method used to determine the Antiviral Index of the present invention is a modification of that described in Sattar I, a test for the virucidal activity of liquid hand washes (rinse-off products). The method is modified in this case to provide reliable data for leave-on products.

The modifications of Sattar I include the product being delivered directly to skin as described below, virus inoculation of the fingerpads as described below, and viral recovery using ten-cycle washing. The inoculated skin site then is completely decontaminated by treating the area with 70% dilution of ethanol in water.

Procedure:

Ten-minute Test:

Subjects (5 per test product) initially wash their hands with a nonmedicated soap, rinse the hands, and allow the hands to dry.

The hands then are treated with 70% ethanol and air dried.

Test product (1.0 ml) is applied to the hands, except for the thumbs, and allowed to dry.

About 10 minutes (±30 seconds) after product application, 10 μl of a Rhinovirus 14 suspension (ATCC VR-284, approximately 1×10⁶ PFU (plaque-forming units)/ml) is topically applied using a micropipette to various sites on the hand within a designated skin surface area known as fingerpads. At this time, a solution of rhinovirus also is applied to the untreated thumb in a similar manner.

After a dry-down period of 7-10 minutes, the virus then is eluted from each of the various skin sites with 1 ml of eluent (Minimal Essential media (MEM)+1% pen-strep-glutamate), washing 10 times per site.

The inoculated skin site then is completely decontaminated by treating the area with a 1:10 dilution of domestic bleach (CLOROX® 5.25% sodium hypochlorite) in tap water, then rinsing with 70% ethanol. Viral titers are determined using standard techniques, i.e., plaque assays or TCID₅₀ (Tissue Culture Infectious Dose).

One-hour test:

Subjects are allowed to resume normal activities (with the exception of washing their hands) between the 1-hour and 3-hour timepoints. After one hour, a rhinovirus suspension is applied to and eluted from designated sites on the fingerpads exactly as described in above for the 10-minute test.

### Example 1

A composition of the invention is prepared by admixing the following ingredients at the indicated weight percentages until homogeneous.

| Ingredient | Weight Percent |
|---|---|
| Triclosan (TCS) | 0.3 |
| Sodium lauryl sulfate | 0.75 |
| Ethanol | 65.0 |
| Sodium xylene sulfonate | 10.0 |
| Fragrance | 0.05 |
| Water | q.s. |

The pH of the composition is about 3.5. The composition has a percent saturation of TCS of about 50%, and excellent antibacterial properties, exhibiting a greater than 1 log reduction in Gram positive and Gram negative bacteria in 30 seconds by the time kill test. The composition also eliminates human rhinovirus from the skin.

The antimicrobial compositions of the present invention have several practical end uses, including hand cleansers, mouthwashes, surgical scrubs, body splashes, antiseptics, disinfectants, hand sanitizer gels, deodorants, dental care additives, mouthwashes, and similar personal care products. Additional types of compositions include foamed compositions, such as creams, mousses, and the like, and compositions containing organic and inorganic filler materials, such as emulsions, lotions, creams, pastes, and the like. The compositions further can be used as an antimicrobial cleanser for hard surfaces, for example, sinks and countertops in hospitals, food service areas, and meat processing plants. The present antimicrobial compositions can be manufactured as dilute ready-to-use compositions, or as concentrates that are diluted prior to use.

The present invention, therefore, encompasses applying an effective amount of the antimicrobial cleansing compositions of the present invention onto nonskin surfaces, such as household surfaces, e.g., countertops, kitchen surfaces, food preparing surfaces (cutting boards, dishes, pots and pans, and the like); major household appliances, e.g., refrigerators, freezers, washing machines, automatic dryers, ovens, microwave ovens, and dishwashers; cabinets; walls; floors; bathroom surfaces, shower curtains, garbage cans, and/or recycling bins, and the like.

The compositions also can be incorporated into a web material to provide an antimicrobial wiping article. The wiping article can be used to clean and sanitize animate or inanimate surfaces.

In one embodiment of the present invention, a person suffering from a rhinovirus cold, or who is likely to be exposed to other individuals suffering from rhinovirus colds, can apply a present antimicrobial composition to his or her hands. This application kills bacteria and inactivates rhinovirus particles present on the hands. Rhinovirus particles therefore are not transmitted to noninfected individuals via hand-to-hand transmission. The amount of the composition applied, the frequency of application, and the period of use will vary depending upon the level of disinfection and cleansing desired, e.g., the degree of microbial contamination and/or skin soiling.

The present antimicrobial compositions provide the advantages of a broad spectrum kill of Gram positive and Gram negative bacteria, and a viral control, in short contact times. The short contact time for a substantial log reduction of bacteria is important in view of the typical 15 to 60 second time frame used to cleanse and sanitize the skin and inanimate surfaces.

The present compositions are effective in short contact time because the antimicrobial agent is present in the aqueous continuous phase of the composition, as opposed to surfactant micelles, and because of the reduced pH of the composition. The antimicrobial agent, therefore, is available to immediately begin reducing bacterial populations, and further is available to deposit on the skin to provide antimicrobial efficacy. In addition, because the antimicrobial agent is in solution as opposed to surfactant micelles, the absolute amount of antimicrobial agent in the composition can be reduced without adversely affecting efficacy, and the antimicrobial agent is not rinsed from the skin with the surfactant prior to performing its antimicrobial function. In addition, the amount of surfactant in the present antimicrobial compositions typically is low, thereby providing additional environmental benefits.

Obviously, many modifications and variations of the invention as hereinbefore set forth can be made without departing from the spirit and scope thereof, and, therefore, only such limitations should be imposed as are indicated by the appended claims.

What is claimed is:

1. A method of reducing a bacteria and a virus population on a surface comprising contacting the surface with a composition for 30 seconds to achieve a log reduction of at least 2 against S. aureus, a log reduction of at least 2.5 against E. coli, and a log reduction of at least 4 against a rhinovirus,
said composition comprising:
(a) about 0.001% to about 5%, by weight, of a phenolic antimicrobial agent;
(b) about 0.1% to 15%, by weight, of a surfactant;
(c) about 2% to about 30%, by weight, of a hydrotrope;
(d) greater than 60% to about 90%, by weight, of a disinfecting alcohol; and
(e) water,
wherein the antimicrobial agent is present in the composition in an amount of at least 25% of saturation concentration, when measured at room temperature.

2. The method of claim 1 further comprising a step of rinsing the composition from the surface.

3. The method of claim 1 wherein the surface is a skin of a mammal.

4. The method of claim 1 wherein the surface is a hard, inanimate surface.

5. The method of claim 1 wherein the composition comprises about 0.01% to about 2%, by weight, of the phenolic antibacterial agent.

6. The method of claim 1 wherein the phenolic antibacterial agent is selected from the group consisting of:
(a) a 2-hydroxydiphenyl compound having the structure 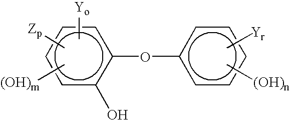
wherein Y is chlorine or bromine, Z is SO₃H, NO₂, or C₁-C₄ alkyl, r is 0 to 3, o is 0 to 3, p is 0 or 1, m is 0 or 1, and n is 0 or 1;
(b) a phenol derivative having the structure 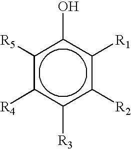
wherein R₁ is hydro, hydroxy, C₁-C₄ alkyl, chloro, nitro, phenyl, or benzyl, R₂ is hydro, hydroxy, C₁-C₆ alkyl, or halo, R₃ is hydro, C₁-C₆ alkyl, hydroxy, chloro, nitro, or a sulfur in the form of an alkali metal salt or ammonium salt, R₄ is hydro or methyl, and R₅ is hydro or nitro;
(c) a diphenyl compound having the structure 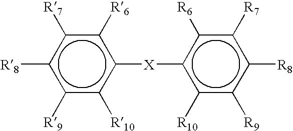
wherein X is sulfur or a methylene group, R₆ and R′₆ are hydroxy, and R₇, R′₇, R₈, R′₈, R₉, R′₉, R₁₀, and R′₁₀, independent of one another, are hydro or halo; and
(d) mixtures thereof.

7. The method of claim 6 wherein the anti-microbial agent comprises triclosan, p-chloro-m-xylenol, or a mixture thereof.

8. The method of claim 1 wherein the anti-microbial agent is present in an amount of at least 50% of saturation concentration.

9. The method of claim 1 wherein the anti-microbial agent is present in an amount of at least 75% of saturation concentration.

10. The method of claim 1 wherein the anti-microbial agent is present in an amount of at least 95% of saturation concentration.

11. The method of claim 1 wherein the surfactant is present in the composition in an amount of about 0.3% to about 10%, by weight of the composition.

12. The method of claim 1 wherein the surfactant comprises an anionic surfactant.

13. The method of claim 1 wherein the surfactant comprises an ampholytic surfactant.

14. The method of claim 1 wherein the surfactant is selected from the group consisting of a C₈-C₁₈ alkyl sulfate, a C₈-C₁₈ alkamine oxide, and mixtures thereof.

15. The method of claim 1 wherein the surfactant comprises a lauryl sulfate, an octyl sulfate, a 2-ethylhexyl sulfate, lauramine oxide, and mixtures thereof.

16. The method of claim 1 wherein the hydrotrope is present in the composition in amount of about 5% to about 20% by weight of the composition.

17. The method of claim 1 wherein the hydrotrope is selected from the group consisting of sodium cumene sulfonate, ammonium cumene sulfonate, ammonium xylene sulfonate, potassium toluene sulfonate, sodium toluene sulfonate, sodium xylene sulfonate, toluene sulfonic acid, xylene sulfonic acid, sodium polynaphthalene sulfonate, sodium polystyrene sulfonate, sodium methyl naphthalene sulfonate, disodium succinate, and mixtures thereof.

18. The method of claim 1 wherein the disinfecting alcohol is present in the composition in an amount of about 65% to about 85%, by weight of the composition.

19. The method of claim 1 wherein the disinfecting alcohol is present in the composition in an amount of about 65% to about 80%, by weight of the composition.

20. The method of claim 1 wherein the disinfecting alcohol comprises a C₁₋₆ alcohol or a mixture thereof.

21. The method of claim 1 wherein the disinfecting alcohol is selected from the group consisting of methanol, ethanol, isopropyl alcohol, n-butanol, n-propyl alcohol, and mixtures thereof.

22. The method of claim 1 wherein the composition further comprises about 0.1% to about 20% by weight of a polyhydric solvent selected from the group consisting of a diol, a triol, and mixtures thereof.

23. The method of claim 22 wherein the polyhydric solvent comprises ethylene glycol, propylene glycol, glycerol, diethylene glycol, dipropylene glycol, tripropylene glycol, hexylene glycol, butylene glycol, 1,2,6-hexanetriol, sorbitol, PEG-4, 1,5-pentanediol, or mixtures thereof.

24. The method of claim 1 wherein the composition has a pH of about 4 to about 9.

25. A method of inactivating viruses and killing bacteria comprising a step of topically applying a composition to a surface in need of such treatment,
said composition comprising:
(a) about 0.001% to about 5%, by weight, of a phenolic antimicrobial agent;
(b) about 0.1% to 15%, by weight, of a surfactant;
(c) about 2% to about 30%, by weight, of a hydrotrope;
(d) greater than 60% to about 90%, by weight, of a disinfecting alcohol; and
(e) water,
wherein the antimicrobial agent is present in the composition in an amount of at least 25% of saturation concentration, when measured at room temperature.

26. The method of claim 25 wherein the surface is animate.

27. The method of claim 25 wherein the surface is inanimate.

28. The method of claim 25 wherein rhinoviruses are inactivated.

29. A method of improving the overall health of a mammal by reducing exposure to viruses and bacteria comprising the steps of:
(a) topically applying a composition to a surface which is prone to viral and/or bacterial contamination; and
(b) allowing the surface to dry,
said composition comprising:
(a) about 0.001% to about 5%, by weight, of a phenolic antimicrobial agent;
(b) about 0.1% to 15%, by weight, of a surfactant;
(c) about 2% to about 30%, by weight, of a hydrotrope;
(d) greater than 60% to about 90%, by weight, of a disinfecting alcohol; and
(e) water,
wherein the antimicrobial agent is present in the composition in an amount of at least 25% of saturation concentration, when measured at room temperature.

30. A method of protecting an individual against infection by rhinoviruses comprising the step of applying a composition to hands of the individual in an amount sufficient to eradicate rhinoviruses, said composition comprising:
(a) about 0.001% to about 5%, by weight, of a phenolic antimicrobial agent;
(b) about 0.1% to 15%, by weight, of a surfactant;
(c) about 2% to about 30%, by weight, of a hydrotrope;
(d) greater than 60% to about 90%, by weight, of a disinfecting alcohol; and
(e) water,
wherein the antimicrobial agent is present in the composition in an amount of at least 25% of saturation concentration, when measured at room temperature.

31. The method of claim 30 wherein the composition is applied prior to the individual being exposed to rhinoviruses.

32. The method of claim 30 wherein the composition is applied multiple times within a twenty-four-hour period.

33. The method of claim 30 wherein the composition is rinsed from the hands.

34. The method of claim 30 wherein the composition is allowed to dry and remain on the hands.

35. An antimicrobial composition comprising:
(a) about 0.001% to about 5%, by weight, of a phenolic antimicrobial agent;
(b) about 0.1% to 15%, by weight, of a surfactant;
(c) about 2% to about 30%, by weight, of a hydrotrope;
(d) greater than 60% to about 90%, by weight, of a disinfecting alcohol; and
(e) water,
wherein the antimicrobial agent is present in the composition in an amount of at least 25% of saturation concentration, when measured at room temperature.
